(19) United States
(12) Patent Application Publication
(10) Pub. No.: US 2011/0054471 A1
(43) Pub. Date: Mar. 3, 2011

(54) Apparatus for Performing an Electrosurgical Procedure

(75) Inventors: Thomas J. Gerhardt, Littleton, CO (US); John J. Kappus, Denver, CO (US); Larry Johnson, Bennett, CO (US); Wayne Siebrecht, Golden, CO (US)

(73) Assignee: TYCO Healthcare Group LP

(21) Appl. No.: 12/548,534

(22) Filed: Aug. 27, 2009

(51) Int. Cl. A61B 18/14

(52) U.S. Cl. 606/51

(57) ABSTRACT

A surgical instrument is provided. The surgical instrument includes a housing having a shaft that extends therefrom that defines a longitudinal axis therethrough. An end effector assembly is operatively connected to a distal end of the shaft and includes a pair of first and second jaw members. The surgical instrument includes a handle assembly that includes a movable handle movable relative to a fixed handle operable to impart movement of the jaw members relative to one another. A rotating assembly is configured for rotating one of the shaft and end effector assembly about the longitudinal axis. The rotating assembly is supported in the housing and includes first and second drive. Each of the first and second drive wheels are selectively movable relative to the housing and configured such that rotation of the first drive wheel causes rotation of the second drive wheel and the shaft.

### BACKGROUND

1. Technical Field

The present disclosure relates to an apparatus for performing an electrosurgical procedure. More particularly, the present disclosure relates to an electrosurgical apparatus that includes a rotating assembly configured to rotate a shaft associated with the electrosurgical apparatus.

2. Description of Related Art

Electrosurgical instruments (e.g., electrosurgical forceps) are well known in the medical arts and typically include a housing, a handle, a shaft and an end effector assembly, which includes jaw members operatively coupled to a distal end of the shaft, that is configured to manipulate tissue (e.g., grasp and seal tissue). Electrosurgical forceps utilize both mechanical clamping action and electrical energy to effect hemostasis by heating the tissue and blood vessels to coagulate, cauterize, seal, cut, desiccate, and/or fulgurate tissue.

In some instances, it may prove advantageous to rotate the shaft and/or the end effector of the electrosurgical forceps, e.g., during an electrosurgical tissue sealing procedure. With this purpose in mind, many electrosurgical forceps may include one or more types of shaft rotation mechanisms and/or devices, such as, for example, a rotating assembly that is in mechanical and/or electromechanical communication with the shaft, housing and/or end effector.

Rotating assemblies are known in the medical art and typically include a rotation wheel that is coaxially connected to a proximal end of a shaft of the electrosurgical instrument. During the manufacturing process of the electrosurgical instrument, design constraints of internal mechanisms associated with the electrosurgical instrument may control location of the rotation wheel placement on the electrosurgical instrument. Because of these design constraints rotation of the shaft is typically a two-handed operation. That is, a clinician may use one hand to hold a handle of the electrosurgical instrument, and the other hand to rotate a rotation wheel of the rotation assembly.

### SUMMARY

The present disclosure provides a forceps that is adapted to connect to a source of electrosurgical energy for performing an electrosurgical procedure. The forceps includes a housing that includes a shaft that extends from the housing and defines a longitudinal axis therethrough. An end effector assembly operatively connects to a distal end of the shaft and includes a pair of first and second jaw members. The first and second jaw members are moveable from an open position wherein the jaw members are disposed in spaced relation relative to one another, to a clamping position wherein the jaw members cooperate to grasp tissue therebetween. The forceps includes a handle assembly including a movable handle movable relative to a fixed handle operable to impart movement of the jaw members relative to one another. The movable handle operatively connects to a drive assembly that together mechanically cooperate to impart movement of the jaw members. The forceps further includes a rotating assembly configured to rotate one of the shaft and the end effector assembly about the longitudinal axis. The rotating assembly is supported in the housing and includes a first drive wheel that defines an axis of rotation substantially perpendicular to the longitudinal axis of the shaft and a second drive wheel defining an axis of rotation substantially parallel to the longitudinal axis of shaft. Each of the first and second drive wheels is selectively movable relative to the housing and configured such that rotation of the first drive wheel causes rotation of the second drive wheel and the shaft.

The present disclosure also provides a surgical instrument configured to manipulate tissue. The surgical instrument includes a housing that includes a shaft that extends from the housing and defines a longitudinal axis therethrough. An end effector assembly operatively connects to a distal end of the shaft and includes a pair of first and second jaw members. The first and second jaw members are moveable from an open position wherein the jaw members are disposed in spaced relation relative to one another, to a clamping position wherein the jaw members cooperate to grasp tissue therebetween. The surgical instrument includes a handle assembly including a movable handle movable relative to a fixed handle operable to impart movement of the jaw members relative to one another. The movable handle operatively connects to a drive assembly that together mechanically cooperate to impart movement of the jaw members. The surgical instrument further includes a rotating assembly configured to rotate one of the shaft and the end effector assembly about the longitudinal axis. The rotating assembly is supported in the housing and includes a first drive wheel that defines an axis of rotation substantially perpendicular to the longitudinal axis of the shaft and a second drive wheel defining an axis of rotation substantially parallel to the longitudinal axis of shaft. Each of the first and second drive wheels is selectively movable relative to the housing and configured such that rotation of the first drive wheel causes rotation of the second drive wheel and the shaft.

### BRIEF DESCRIPTION OF THE DRAWING

Various embodiments of the present disclosure are described hereinbelow with references to the drawings, wherein:

FIG. 1 is a side, perspective view of an endoscopic bipolar forceps showing a housing including a rotation assembly, a shaft and an end effector assembly according to an embodiment of the present disclosure;

FIG. 2 is a left, side view of the housing and the rotation assembly of the endoscopic bipolar forceps illustrated in FIG. 1;

FIG. 3A is a right, internal view of the various components of the rotation assembly illustrated in FIG. 2;

FIG. 3B is a front view of the bipolar forceps illustrated in FIG. 2; and

FIG. 4 is a side, perspective view of the internal components of the rotation assembly illustrated in FIG. 2 according to an alternate embodiment of the present disclosure.

### DETAILED DESCRIPTION

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

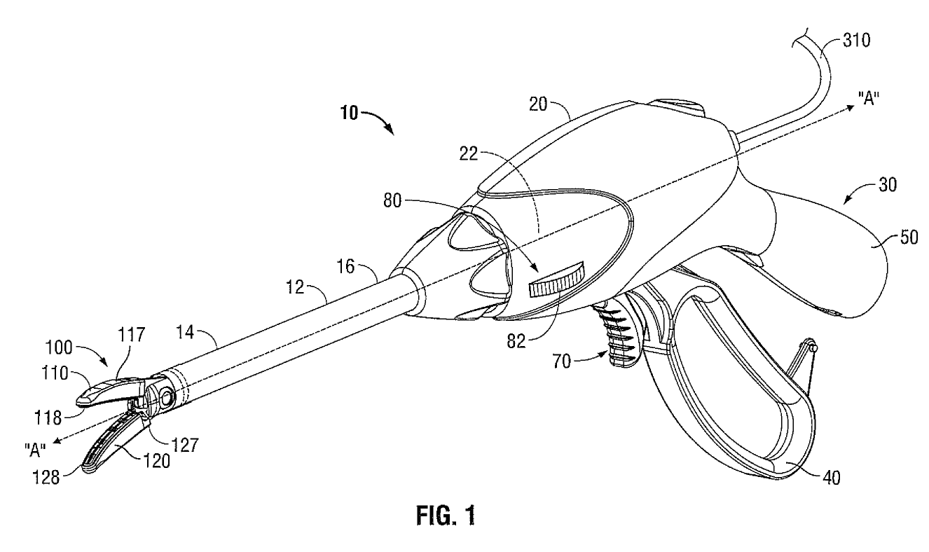

With reference to FIG. 1, an illustrative embodiment of an electrosurgical apparatus (e.g., bipolar forceps 10) for performing an electrosurgical procedure is shown. Bipolar forceps 10 is operatively and selectively coupled to an electrosurgical generator (not shown) for performing an electrosurgical procedure. As noted above, an electrosurgical procedure may include sealing, cutting, cauterizing coagulating, desiccating, and fulgurating tissue all of which may employ RF energy. The generator may be configured for monopolar and/or bipolar modes of operation. The generator may include or is in operative communication with a system (not shown) that may include one or more processors in operative communication with one or more control modules that are executable on the processor. A control module (not explicitly shown) may be configured to instruct one or more modules to transmit electrosurgical energy, which may be in the form of a wave or signal/pulse, via one or more cables (e.g., a cable 310) to one or both seal plates 118, 128.

With reference again to FIG. 1, bipolar forceps 10 is shown for use with various electrosurgical procedures and generally includes a housing 20, an electrosurgical cable 310 that connects the forceps 10 to a source of electrosurgical energy (e.g., electrosurgical generator not shown), a handle assembly 30, a rotating assembly 80, a trigger assembly 70, a drive assembly (not shown), and an end effector assembly 100 that operatively connects to the drive assembly. The drive assembly may be in operative communication with handle assembly 30 for imparting movement of one or both of a pair of jaw members 110, 120 of end effector assembly 100. End effector assembly 100 includes opposing jaw members 110 and 120 (FIG. 1) that mutually cooperate to grasp, seal and, in some cases, divide large tubular vessels and large vascular tissues.

Forceps 10 includes a shaft 12 that has a distal end 14 configured to mechanically engage the end effector assembly 100 and a proximal end 16 that mechanically engages the housing 20. In the drawings and in the descriptions that follow, the term “proximal,” as is traditional, will refer to the end of the forceps 10 that is closer to the user, while the term “distal” will refer to the end that is farther from the user.

With continued reference to FIG. 1, handle assembly 30 includes a fixed handle 50 and a movable handle 40. Fixed handle 50 is integrally associated with housing 20 and handle 40 is movable relative to fixed handle 50. Fixed handle 50 may include one or more ergonomic enhancing elements to facilitate handling, e.g., scallops, protuberances, elastomeric material, etc.

Movable handle 40 of handle assembly 30 is ultimately connected to the drive assembly, which together mechanically cooperate to impart movement of one or both of the jaw members 110 and 120 to move from an open position, wherein the jaw members 110 and 120 are disposed in spaced relation relative to one another, to a clamping or closed position, wherein the jaw members 110 and 120 cooperate to grasp tissue therebetween.

End effector assembly 100 includes opposing jaw members 110 and 120 that are coupled to shaft 12. Jaw members 110, 120 may be operatively and pivotably coupled to each other and located adjacent the distal end 14 of shaft 12.

Jaw member 110 includes an insulative jaw housing 117 and an electrically conductive seal plate 118. The insulative housing 117 is configured to securely engage the electrically conductive seal plate 118. This may be accomplished by stamping, by overmolding, by overmolding a stamped electrically conductive sealing plate and/or by overmolding a metal injection molded seal plate. All of these manufacturing techniques produce an electrode having a seal plate 118 that is substantially surrounded by the insulating substrate. Within the purview of the present disclosure, jaw member 110 may include a jaw housing 117 that is integrally formed with a seal plate 118.

Jaw member 120 includes a similar structure having an outer insulative housing 127 that may be overmolded to capture seal plate 128.

For a more detailed description of the bipolar forceps 10 including end effector 100, handle assembly 30 including movable handle 40, and electrosurgical cable 310 (including line-feed configurations and/or connections), reference is made to commonly owned U.S. application Ser. No. 10/369,894.

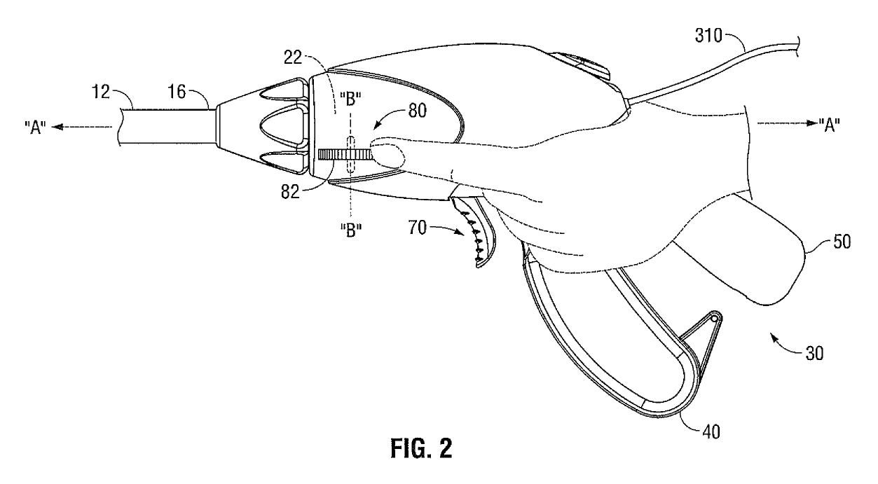

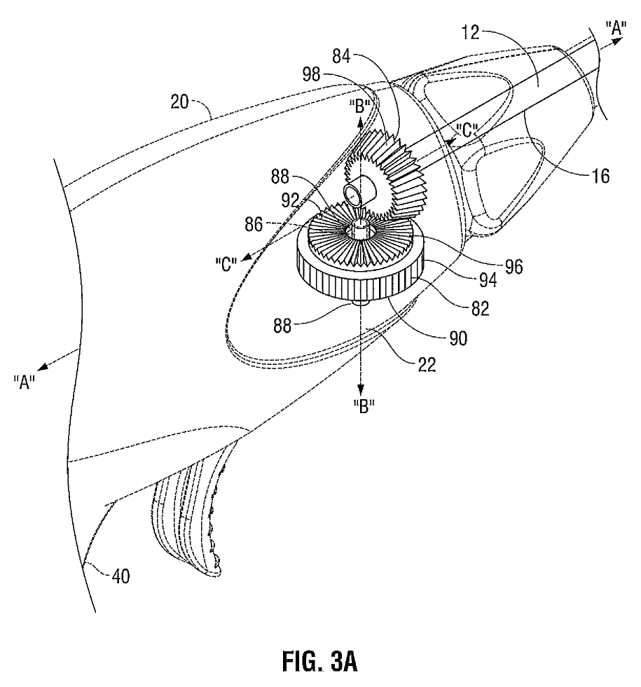

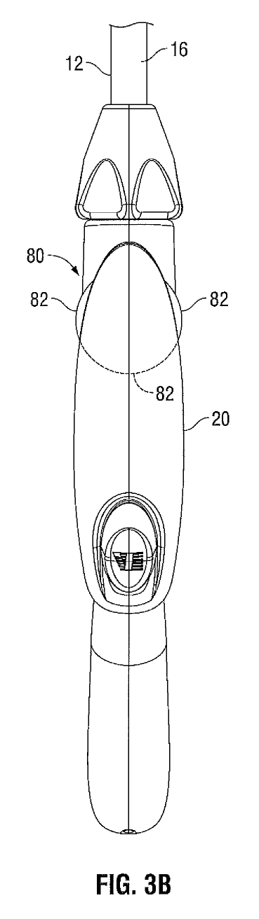

With reference to FIGS. 1-3B, and initially with reference to FIG. 1, an embodiment of a rotating assembly 80 configured for use with the bipolar forceps 10 is shown. Rotating assembly 80 and operative components and/or members associated therewith may be formed from any suitable material including but not limited to injection moldable plastics, such as, for example, acrylonitrile butadiene styrene (ABS), Polycarbonates (Poly Carb), or other suitable material. The rotating assembly 80 of the present disclosure allows rotation of the shaft 12 via an index finger of a hand that holds a handle, e.g., fixed handle 50, of the bipolar forceps 10. To this end, the rotating assembly 80 includes a first drive wheel or finger knob 82 in the form of a gear wheel that is configured to receive a finger (e.g., index finger, thumb, etc.) of a hand that holds the fixed handle 50. Rotation of shaft 12 is achieved by either “pushing” the drive wheel 82 forward (i.e., moving the drive wheel 82 in a counter-clockwise direction) or “pulling” the drive wheel 82 backward (i.e., moving the drive wheel 82 in a clockwise direction). The first drive wheel 82 operably engages to a second drive wheel 84 (FIG. 3A) also in the form of a gear wheel that operably engages the proximal end 16 of the shaft 12.

With reference to FIG. 3A, first drive wheel 82 is disposed at a predetermined position within cavity 22 of housing 20 and is shown associated with the internal cavity 22 of the housing 20. In the embodiment illustrated in FIGS. 1-3B, rotating assembly 80 includes an axle 86 that supports the first drive wheel 82. Axle 86 operably couples to the drive wheel 82 and provides a central axis of rotation for the drive wheel 82.

In some embodiments, the drive wheel 82 is fixedly coupled to the axle 86 (i.e., moves with the axle 86) such that the drive wheel 82 and the axle 86 rotate simultaneously. In this instance, the axle 86 is rotatably supported within housing 20 and moveable relative to the housing 20. More particularly, axle 86 couples to the drive wheel 82 and seats within corresponding bores or holes 88 operatively associated within the cavity 22 of housing 20. In this embodiment, the axle 86 is configured to rotate within the holes 88 when the drive wheel 82 is “pushed” or “pulled”.

In an alternate embodiment, the drive wheel 82 may be rotatably coupled to the axle 86 (i.e., moves relative to the axle 86). In this embodiment, axle 86 extends through a central aperture of the drive wheel 82 and is fixedly attached within the cavity 22 of housing 20. In this embodiment, the drive wheel 82 may include one or more configurations of bearing such as, for example, bushing, rolling element bearing, jewel bearing, fluid bearing, magnetic hearing, flexure bearings, and/or other suitable structure(s) that are configured to facilitate rotation of the drive wheel 82 with respect to the axle 86. In this embodiment, the axle 86 is configured not to rotate when the drive wheel 82 is “pushed” or “pulled.”

The specific mechanical relationships and/or configurations between drive wheel 82 and axle 86 will depend on the ultimate needs of a manufacturer and/or user. As can be appreciated by one skilled in the art, other suitable drive wheel 82 and axle 86 configurations and/or combinations are possible and contemplated herein.

As noted above, drive wheel 82 may be in the form of a gear wheel. More particularly, drive wheel 82 is in the form of a bevel gear that includes a generally circumferential (e.g., conical) configuration. Drive wheel 82 includes a bottom, toothless surface 90 and a top, tooth bearing surface 92, see FIG. 3A. Drive wheel 82 includes a generally circumferential sidewall 94 that is accessible by one or more fingers of a hand. Sidewall 94 may have a textured or rubber-like surface to facilitate rotation, especially under wet surgical conditions. In the illustrated embodiment, sidewall 94 is shown having a knurled configuration.

Tooth bearing surface 92 includes a plurality of teeth 96 that are configured to mesh with a plurality of teeth or cogs 98 associated with the second drive wheel 84. In the embodiment illustrated in FIGS. 1-3B, the drive wheel 82 is an external bevel gear. That is, the plurality of teeth 96 is configured to point outward. Alternatively, the drive wheel 82 may be configured as an internal bevel gear, wherein the plurality of teeth is configured to point inward.

Second drive wheel 84 is substantially similar to first drive wheel 82. Accordingly, only those features and/or operative components that are unique or distinctive to second drive wheel 84 will be described herein.

Second drive wheel 84 is positioned at a predetermined position within cavity 22 of housing 20 and is associated with the internal cavity 22 of the housing 20 and the shaft 12. More particularly, second drive wheel 84 is fixedly coupled or mounted to the proximal end 16 of shaft 12 by suitable coupling methods, such as, for example, brazing, welding, soldering, snap-fit, tongue and groove, etc. In some embodiments, second drive wheel 84 and proximal end 16 may be unitary component (e.g., overmolding, injection molding, etc).

The first drive wheel 82 defines a central axis of rotation “B” and second drive wheel 84 defines a central axis of rotation “C”. The central axis of rotation “B” of first drive wheel 82, central axis of rotation “C” of second drive wheel 84 and the longitudinal axis “A” defined by shaft 12 may be oriented relative to one another by any suitable angle. In this instance, the relative angle of the gears is configured accordingly to compensate for the angle. In the embodiments illustrated in FIGS. 1-3B, longitudinal axis “A” defined by shaft 12 and central axis of rotation “C” defined by second drive wheel 84 are oriented parallel to each other and perpendicular to the central axis of rotation “B” of first drive wheel 82. By altering the orientation of one or both of the axes “B” and “C”, and thus altering the gear interfaces of the first and second drive wheels, 82, 84, respectively, the direction of motion of first and second drive wheels 82, 84, respectively, and/or shaft 12 can be reversed. That is to say, the first drive wheel 82 may be oriented with respect to the second drive wheel 84 and the shaft 12 at an angle that ranges from about 0° to about 90°.

First drive wheel 82 and second drive wheel 84 may be configured to meet specific rotation, torque, and/or speed requirements of the shaft. To this end, first and second drive wheels 82, 84 may include any suitable gear ratio. More specifically, first and second drive wheels 82, 84 may be equally sized (e.g., have the same diameter) or unequally sized (e.g., have different diameters). Moreover, the first and second drive wheels 82, 84 may have the same or different amount of teeth. The specific gear configuration of first and second drive wheels 82, 84, respectively, will depend on the ultimate needs of a manufacturer and/or user. For example, in certain instances, the gear ratio of the first and second drive wheels 82, 84, respectively, may be varied to, for instance, “gear down” for finer rotational control or movement of the shaft.

First drive wheel 82 and second drive wheel 84 may be configured to rotate shaft 12 in a counter-clockwise or clockwise direction. More particularly, in the embodiment illustrated in FIGS. 1-3B, clockwise rotation of first drive wheel 82 causes counter-clockwise rotation of the second drive wheel 84 and shaft 12 and/or end effector assembly 100 including first and second jaw members 110, 120, respectively, while counter-clockwise rotation of first drive wheel 82 causes clockwise rotation of the second drive wheel 84 and shaft 12 and/or end effector assembly 100 including first and second jaw members 110, 120, respectively.

While first drive wheel 82 and second drive wheel 84 of the rotating assembly 80 are described herein as including a bevel gear configuration, it is within the purview of the present disclosure that the first drive wheel 82 and second drive wheel 84 of the rotating assembly 80 employ other suitable gear configurations. For example, spur gears, helical gears, double helical gears, hypoid gears, worm gears, etc. may all be employed with first drive wheel 82 and second drive wheel 84 of the rotating assembly 80 of the present disclosure.

In some instances, it may prove useful to rotate the drive wheel 82 via a thumb, while in some instances it may prove useful to rotate the drive wheel 82 via the index finger. To this end, drive wheel 82 may be accessible from either side of the housing. More particularly, the drive wheel 82 may extend through both the right and left sides of the housing 20 (see FIG. 3B) so that the first drive wheel 82 may be accessible by one or more fingers of a hand (e.g., either a right or left hand) that grasps the handle of the bipolar forceps 10. First drive wheel 82 may also be configured to be accessible from either the left or right side of the housing 20. In this instance, first drive wheel 82 extends partially through the housing 20.

In use, a user may grasp movable handle 40 of handle assembly 30. Prior to or while tissue is grasped between the first and second jaw members 110, 120, respectively, a user may rotate first driving wheel 82. This rotation of first drive wheel 82 causes the plurality of teeth 96 of the first drive wheel 82 to mesh with the plurality of teeth 98 of the second drive wheel 84, which, in turn, causes second drive wheel 84 to rotate which causes the shaft 12 and/or first and second jaw members 110, 120 to rotate.

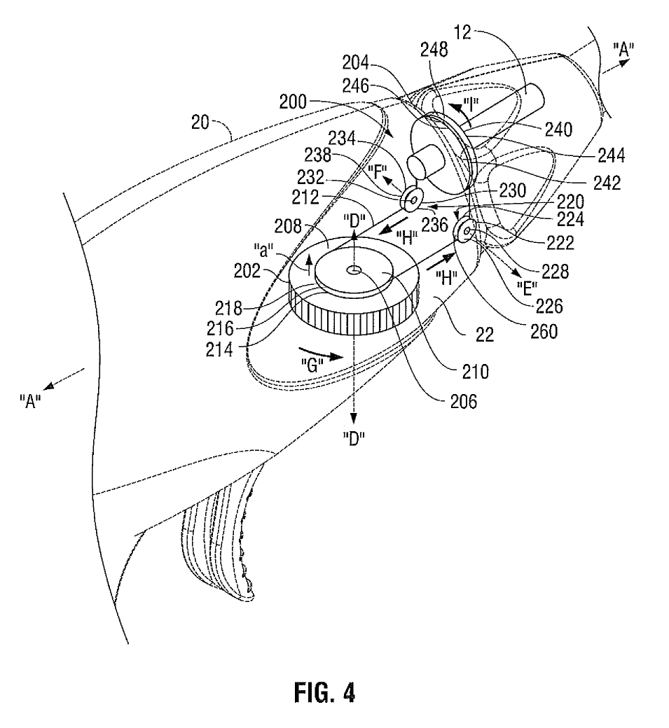

With reference now to FIG. 4, an alternate embodiment of a rotating assembly is shown generally as 200 and described. In order to achieve the same rotation of shaft 12, rotating assembly 200 may be configured as a pulley and taut belt or cord system. To this end, rotating assembly 200 includes a first driving wheel 202 that is in mechanical communication with a second driving wheel 204.

First driving wheel 202 is disposed at a predetermined position within cavity 22 of housing 20 and is associated with the internal cavity 22 of the housing 20. First driving wheel 202 may be operably coupled within internal cavity 22 of housing 20 by any of the aforementioned methods. More particularly, an axle 206 that is configured substantially similar to axle 86 operably couples first drive wheel 202 to the housing 20. Axle 206 and first drive member 202 are configured to function and operate in a manner substantially similar to that of first drive member 82 and axle 86 and, as a result thereof, will only be described to the extent necessary to explain the operational and functional difference with respect to the embodiments illustrated in FIGS. 3A and 4.

First drive wheel 202 defines a central axis of rotation “D” that is perpendicular to the longitudinal axis “A” defined by the shaft 12. First drive wheel 202 includes a top surface 208. Located on top surface 208 may be one or more pulley structures 210. In the embodiment illustrated in FIG. 4, pulley structure 210 includes a generally circumferential configuration and extends from a plane “a” of top surface 208 of drive wheel 202. Pulley structure 210 includes a bottom flange 214 and a top flange 216. Bottom flange 214 abuts top surface 208 of first drive wheel 202. A circumferential groove or channel 218 is located between the bottom and top flanges 214, 216, respectively. Groove 218 is configured to receive a belt, cable, band, or cord 212 such that a frictional interface between the belt 212 and groove 218 is achieved. To gain a desired mechanical advantage, groove 218 and belt 212 may be formed from or include materials that have a relatively high coefficient of friction. As can be appreciated by one skilled in the art, the higher the coefficient of friction between the surfaces of the materials that the groove 218 and belt 212 are formed from, the less likely there will be “slipping” between the groove 218 and the belt 212 when either the groove 218 or belt 212 are moved with respect to each other.

With continued reference to FIG. 4, rotating assembly 200 includes one or more structures configured to transmit the rotational force of the first drive wheel 202 to the second drive wheel 204. To this end, a pair of idler pulleys 220 is in mechanical communication with each of the first and second drive wheels 202, 204, respectively. More particularly, a first idler pulley 222 operably couples to the internal cavity 22 of housing 20. First idler pulley 222 may be configured substantially similar to first drive wheel 202. More particularly, first idler pulley 222 is disposed at a predetermined position within internal cavity 22 of housing 20. An axle 228 extends through the idler pulley 222. First idler pulley 222 includes an axis of rotation “E” that is substantially perpendicular to the central axis of rotation “D” of the first drive wheel 202 and the longitudinal axis “A” defined by the shaft 12. First idler pulley 222 includes a pair of left and right flanges 224, 226, respectively. Located between left and right flanges 224, 226, respectively, is a circumferential groove or channel 260. Groove 260 is configured to receive the belt 212. As noted above, the pair of idler pulleys 220 is configured to transmit the rotational force of the first drive wheel 202 to the second drive wheel 204. With this purpose in mind, idler pulley 222 may be disposed within internal cavity 22 of housing 20 so that the belt 212 extends parallel with respect to the plane “a” of top surface 208 of drive wheel 202 to the idler pulley 222. Moreover, and as best seen in FIG. 4, idler pulley 222 maintains belt 212 in a generally perpendicular orientation with respect to the plane “a” of top surface 208 of first drive wheel 202 when the belt 212 is looped around the idler pulley 222. This configuration of idler pulley 222 and drive wheel 202 minimizes the “drag” of the belt 212 when first drive wheel 202 is moved, e.g., in a clockwise direction, and facilitates the rotation of the first drive wheel 202. As noted above with respect to the interaction between the first drive wheel 202 and belt 212, in some instances it may prove useful to provide a frictional interface between the belt 212 and groove 260.

A second idler pulley 230 operably couples to the internal cavity 22 of housing 20. Second idler pulley 230 may be configured substantially similar to first idler pulley 222 and/or first drive wheel 202. More particularly, second idler pulley 230 is disposed at a predetermined position within internal cavity 22 of housing 20. An axle 232 extends through the second idler pulley 230. Second idler pulley 230 includes a axis of rotation “F” that is aligned along the same axis of rotation “E” of first idler pulley 222 and substantially perpendicular to the central axis of rotation “D” of the first drive wheel 202 and the longitudinal axis “A” defined by the shaft 12. Second idler pulley 230 includes a pair of left and right flanges 234, 236, respectively. Located between left and right flanges 234, 236, respectively, is a circumferential groove or channel 238. Groove 238 is configured to receive the belt 212. As noted above, the pair of idler pulleys 220 is configured to transmit the rotational force of the first drive wheel 202 to the second drive wheel 204. More specifically, and as best seen in FIG. 4, idler pulley 230 is configured to maintain belt 212 in a generally perpendicular orientation with respect to the plane “a” of top surface 208 of first drive wheel 202 when the belt 212 is looped around the idler pulley 230. As with the first idler pulley 222, idler pulley 230 may be disposed within internal cavity 22 of housing 20 such that the belt 212 extends parallel with respect to the plane “a” of top surface 208 of drive wheel 202 to the idler pulley 230. This configuration of idler pulley 230 and drivel wheel 202 minimizes the “drag” of the belt 212 when first drive wheel 202 is moved, e.g., in a clockwise direction, and facilitates the rotation of the first drive wheel 202. As noted above with respect to the interaction between the first drive wheel 202 and belt 212, in some instances it may prove useful to provide a frictional interface between the belt 212 and groove 260.

While each of the idler pulleys 222, 230 has been described herein having its own separate axles, 228 238, respectively, it is within the purview of the present disclosure that a common axle extends through each of the idler pulleys 222, 230. A common axle may extend laterally within the internal cavity 22 of the housing 20 from a left side of the housing 20 to a right side of the housing 20. A common axle configured in this manner provides additional structural support for each of the idler pulleys 222, 230.

Second drive wheel 204 is configured to function and operate in a manner substantially similar to that of second drive member 84 and will only be described to the extent necessary to explain the operational and functional difference with respect to the embodiments illustrated in FIGS. 3A and 4. Second drive wheel 204 operably couples to the shaft 12 or portion thereof. Second drive wheel 204 includes a front surface 240 and a rear surface 242. Second drive wheel includes a front flange 244 and a rear flange 246. In the embodiment illustrated in FIG. 4, front flange 244 operably couples to the shaft 12. While FIG. 4 depicts the shaft 12 extending through the second drive wheel 204, it is within the purview of the present disclosure that the shaft does not extend through the second drive wheel 204; this of course will depend on the contemplated needs of a manufacturer and/or user. A circumferential groove or channel 248 is located between the front and rear flanges 244, 246, respectively. Groove 248 is configured to receive the belt 212 such that a frictional interface between the belt 212 and groove 248 is achieved. To gain a desired mechanical advantage, groove 248 and belt 212 may be formed from or include materials that have a relatively high coefficient of friction. As noted above with respect to groove 218 and belt 212, the higher the coefficient of friction between the surfaces of the materials that the groove 248 and belt 212 are formed from the less likely there will be “slipping” between the groove 248 and the belt 212 when either the groove 248 or belt 212 are moved with respect to each other.

In use, a user may grasp movable handle 40 of handle assembly 30. Prior to or while tissue is grasped between the first and second jaw members 110, 120, respectively, a user may rotate first driving wheel 202 in the direction indicated by directional arrow “G” (i.e., counter-clockwise direction). This rotation of first drive wheel 202 causes the belt 212 to move in the direction indicated by directional arrows “H” and around and/or within the groove 260 of first idler pulley 222 which, in turn, causes second drive wheel 204 to rotate in the direction indicated by directional arrow “I” (i.e., counter-clockwise direction) and around and/or within groove 238 of second idler pulley 230 which ultimately causes the shaft 12 and/or first and second jaw members 110, 120 to rotate.

From the foregoing and with reference to the various figure drawings, those skilled in the art will appreciate that certain modifications can also be made to the present disclosure without departing from the scope of the same. For example, in some embodiments, the rotational assemblies 80, 200 of the present disclosure may employ a cog interface in which the first and second drive wheels are configured as post type features that intersect one another at a desired angle.

The rotating assembly 80, 200 may employ a ratchet and pawl system. More particularly, a first drive wheel may be configured as a linear sliding lever or switch that protrudes from the handle or housing. In this embodiment, when the sliding lever is moved (e.g., in an inward or outward direction) a ratcheting effect would cause the shaft to rotate.

It is contemplated that any of the previously described embodiments of the rotating assemblies 80, 200 may include an electromechanical interface between the first and second drive wheels and/or the shafts. More particularly, one or more types of solenoids and/or servos may be in electromechanical communication with the first and second drive wheels 82, 84, respectively, and shaft 12. Likewise, one or more types of solenoids and/or servos may be in electromechanical communication with the first and second drive wheels 202, 204, respectively, and shaft 12.

It is contemplated that any of the aforementioned embodiments of the rotating assemblies 80, 200 may include one or more springs or other suitable biasing member(s) that is configured to maintain any of the aforementioned first and/or second drive wheels, e.g., drive wheel 82, and/or shaft 12 in a specific position or orientation, e.g., maintain shaft 12 in a initial non-rotated position such that the jaw members 110, 120 are an upright position, as best seen in FIG. 1)

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A forceps, comprising:
a housing having a shaft that extends therefrom that defines a longitudinal axis therethrough;
an end effector assembly operatively connected to a distal end of the shaft and having a pair of first and second jaw members, the first and second jaw members moveable from an open position wherein the jaw members are disposed in spaced relation relative to one another, to a clamping position wherein the jaw members cooperate to grasp tissue therebetween;
a handle assembly including a movable handle movable relative to a fixed handle operable connected to impart movement of the jaw members relative to each other; and
a rotating assembly for rotating the shaft and the end effector assembly about the longitudinal axis, the rotating assembly supported in the housing and including a first drive wheel defining an axis of rotation substantially perpendicular to the longitudinal axis of the shaft and a second drive wheel defining an axis of rotation substantially parallel to the longitudinal axis of shaft,
wherein each of the first and second drive wheels are selectively movable relative to the housing and configured such that rotation of the first drive wheel causes rotation of the second drive wheel and the shaft.

2. The forceps of claim 1, wherein the second drive wheel of the rotating assembly is operatively coupled to the shaft.

3. The forceps of claim 1, wherein each of the first and second drive wheels of the rotating assembly includes a beveled gear.

4. The forceps of claim 1, wherein the first drive wheel is accessible from a left and right side of the housing.

5. The forceps of claim 1, wherein the first drive wheel is knurled.

6. The forceps of claim 1, wherein the first drive wheel is oriented with respect to the second drive wheel at an angle that ranges from about 0° to about 90°.

7. The forceps of claim 1, wherein the rotating assembly includes at least one axle that is in mechanical communication with the first drive wheel that is movable relative to the axle.

8. The forceps of claim 7, wherein the drive wheel includes a bearing configuration to facilitate rotation of the first drive wheel with respect to the axle.

9. The forceps of claim 8, wherein the bearing configuration is selected from the group consisting of bushing, rolling element bearing, jewel bearing, fluid bearing, magnetic bearing and flexure bearings.

10. The forceps of claim 7, wherein the first drive wheel is fixedly coupled to the axle such that the first drive wheel and the axle rotate simultaneously.

11. The forceps of claim 10, wherein the axle is rotatably coupled to the housing and moveable relative thereto.

12. The forceps of claim 11, wherein the axle is coupled to the drive wheel and seats in opposite sides thereof within corresponding bores operatively associated within the cavity of housing.

13. A forceps, comprising:
a housing having a shaft that extends therefrom that defines a longitudinal axis therethrough;
an end effector assembly operatively connected to a distal end of the shaft and having a pair of first and second jaw members, the first and second jaw members moveable from an open position wherein the jaw members are disposed in spaced relation relative to one another, to a clamping position wherein the jaw members cooperate to grasp tissue therebetween;
a handle assembly including a movable handle movable relative to a fixed handle operable connected to impart movement of the jaw members relative to each other; and
a rotating assembly for rotating the shaft and the end effector assembly about the longitudinal axis, the rotating assembly supported in the housing and including a first drive wheel defining an axis of rotation substantially perpendicular to the longitudinal axis of the shaft and a second drive wheel defining an axis of rotation substantially parallel to the longitudinal axis of shaft,
wherein each of the first and second drive wheels are selectively movable relative to the housing and configured such that rotation of the first drive wheel causes rotation of the second drive wheel and the shaft.

14. The forceps of claim 13, wherein the first drive wheel includes a top surface that includes a pulley structure that includes top and bottom flanges with a circumferential groove defined therebetween.

15. The forceps of claim 14, wherein the second drive wheel includes front and rear flanges with a circumferential groove defined therebetween.

16. The forceps of claim 15, wherein the first and second drive wheels are in operative communication with each other via a belt that is movable within the circumferential grooves of the first and second drive wheels.

17. The forceps of claim 16, wherein the rotating assembly further includes at least one idler pulley configured to convert a rotational force of the first drive wheel to the second drive wheel.

18. The forceps of claim 17, wherein the belt extends from the first drive wheel in a plane that is parallel to the top surface of the first drive wheel.

19. A surgical instrument configured to manipulate tissue, the surgical instrument comprising:
a housing having a shaft that extends therefrom that defines a longitudinal axis therethrough;
an end effector assembly operatively connected to a distal end of the shaft and having a pair of first and second jaw members, the first and second jaw members moveable from an open position wherein the jaw members are disposed in spaced relation relative to one another, to a clamping position wherein the jaw members cooperate to grasp tissue therebetween;
a handle assembly including a movable handle movable relative to a fixed handle operable to impart movement of the jaw members relative to one another; and
a rotating assembly for rotating the shaft and the end effector assembly about the longitudinal axis, the rotating assembly being supported in the housing and including a first drive wheel defining an axis of rotation substantially perpendicular to the longitudinal axis of the shaft and a second drive wheel defining an axis of rotation substantially parallel to the longitudinal axis of shaft,
wherein each of the first and second drive wheels are selectively movable relative to the housing, wherein each of the first and second drive wheels includes a toothless surface and tooth bearing surface, the plurality of teeth associated with the first drive wheel configured to mesh with the plurality of teeth associated with the second drive wheel such that rotation of the first drive wheel causes rotation of the second drive wheel and the shaft.

20. The surgical instrument of claim 18, wherein the second drive wheel of the rotating assembly is operatively coupled to the shaft and wherein each of the first and second drive wheels of the rotating assembly is a beveled gear.
